(19) United States
(12) Patent Application Publication
(10) Pub. No.: US 2017/0028032 A1
(43) Pub. Date: Feb. 2, 2017

(54) METHOD FOR MASKING BITTERNESS OF COMPOSITION CONTAINING COLLAGEN PEPTIDE

(71) Applicant: SUNTORY HOLDINGS LIMITED, Osaka (JP)

(72) Inventors: Nozomi KITAHARA, Osaka (JP); Megumi OKADA, Osaka (JP)

(73) Assignee: SUNTORY HOLDINGS LIMITED, Osaka (JP)

(21) Appl. No.: 15/302,519

(22) PCT Filed: Apr. 6, 2015
(86) PCT No.: PCT/JP2015/060729

§ 371 Date: Oct. 7, 2016

(30) Foreign Application Priority Data
Apr. 10, 2014 (JP) 2014-081254

(51) Int. Cl. A61K 38/39; A23L 27/00; A23L 33/18; A61K 47/24; A61K 9/00

(52) U.S. Cl. CPC A61K 38/39; A61K 47/24; A61K 9/0053; A23L 33/18; A23L 27/84; A23V 2002/00

(57) ABSTRACT

An object of the present invention is to provide a technique for masking the bitterness of collagen peptide containing compositions.

By incorporating glycerophospholipids comprising neutral glycerophospholipids and acidic glycerophospholipids in specified proportions or incorporating sphingoglycolipids in a specified proportion relative to collagen peptides, there can be provided methods for masking the bitterness of collagen peptide containing compositions.

### TECHNICAL FIELD

The present invention relates to methods for masking the bitterness of compositions containing collagen peptides. More specifically, the present invention relates to an invention wherein the distinctive bitterness of collagen peptides is masked by incorporating glycerophospholipids or sphingoglycolipids.

### BACKGROUND ART

Recently, collagen has been reported to have various physiological effects such as a bone reinforcing effect that leads to prevention and/or amelioration of osteoporosis, an effect for promoting the metabolism of a living tissue by reversing its lowered function due to aging, a skin metabolism promoting effect, a skin activating effect, and anti-aging effect for the skin with a view to preventing and/or improving wrinkles; having these effects, collagen is widely used not only as a raw material in cosmetics, foods and beverages but also as a biological, functional material for use in pharmaceuticals. The decomposition product of collagen is known to be broken down to amino acids, dipeptides or tripeptides as it is digested and absorbed, and among them the dipeptides or tripeptides have been shown to be effective as shown above. Further, it has been reported that collagen-derived hydroxyproline (Hyp) containing dipeptides (such as Pro-Hyp (PO) and Hyp-Gly (OG)) and tripeptides, when acted on dermal fibroblasts, activate their proliferation to promote the production of collagen and hyaluronic acid (Non-Patent Document 1 and Patent Document 1).

However, it is known that collagen as a naturally occurring protein has a distinctive unpleasant smell and taste while many amino acids and peptides as decomposition products of the protein have bitterness (Patent Document 2). Therefore, when using collagen and collagen peptides as raw materials of foods and beverages, they could be a factor that makes daily and continuous oral ingestion difficult.

To deal with this problem, methods for reducing the distinctive unpleasant smell and taste of collagen have been extensively studied and are exemplified by methods that involve adding ethyl octanoate (Patent Document 3), phenyl ethyl methyl ether (Patent Document 4), dietary fiber (Patent Document 5) or L-lactic acid (Patent Document 6). Methods that have so far been studied for reducing the bitterness of peptides include a method that involves adding acidic phospholipids and their lyso forms (Patent Document 2).

### CITATION LIST

### Patent Literature

Patent Document 1: Japanese Patent No. 4995155
Patent Document 2: JPH 08-173093 A
Patent Document 3: JP 2006-197856 A
Patent Document 4: JP 2008-167672 A
Patent Document 5: JP 2010-104338 A
Patent Document 6: JP 2013-42667 A

### Non-Patent Literature

Non-Patent Document 1: Journal of Dermatological Science, July 2007, Vol. 47, p. 102, 179

### SUMMARY OF INVENTION

### Technical Problem

As described above, collagen and collagen peptides with smaller weight average molecular weights have been verified to possess various useful effects, so a further technological development is desired for suppressing the bitterness of such collagen and collagen peptides to enable their daily and continuous ingestion.

An object, therefore, of the present invention is to provide a technique for masking the bitterness of collagen peptide containing compositions.

### Solution to Problem

The present inventors conducted an intensive study with a view to solving the above-mentioned problem and found as a result that by incorporating glycerophospholipids in a collagen peptide containing composition, wherein the content of a neutral glycerophospholipid is at least 1.5 times the content of an acidic glycerophospholipid on a weight basis in the glycerophospholipids, or by incorporating sphingoglycolipid(s) in a collagen peptide containing composition, wherein the sphingoglycolipid(s) are present in 0.01 to 100 parts by weight per 100 parts by weight of the collagen peptides, it is possible to sufficiently mask the bitterness of collagen peptides in the collagen peptide containing composition, and to enable the composition to be ingested freely and easily. This finding has led to the accomplishment of the present invention.

The present invention encompasses but is not limited to the following modes.

(1) A method for masking the bitterness of a collagen peptide containing composition, which comprises incorporating glycerophospholipids in the composition, wherein:

the sum contents of Pro-Hyp and Hyp-Gly relative to the total amount of the collagen peptides contained in the collagen peptide containing composition are 0.01 to 10 wt %, and

in the glycerophospholipids, the content of a neutral glycerophospholipid is at least 1.5 times the content of an acidic glycerophospholipid on a weight basis.

(2) The method according to (1) wherein the collagen peptides have a weight average molecular weight of less than 5000.
(3) The method according to (1) or (2) wherein the neutral glycerophospholipid is incorporated in 0.01 to 100 parts by weight per 100 parts by weight of the collagen peptides.
(4) The method according to any one of (1) to (3) wherein the neutral glycerophospholipid is selected from the group consisting of phosphatidyl choline, phosphatidyl ethanolamine, and a combination thereof.
(5) The method according to any one of (1) to (4) wherein the neutral glycerophospholipid is derived from a ceramide raw material and a ceramide raw material is incorporated in 0.01 to 200 parts by weight per 100 parts by weight of the collagen peptides.
(6) The method according to (5) wherein the ceramide raw material is milk-derived ceramide.
(7) A method for masking the bitterness of a collagen peptide containing composition, which comprises incorporating sphingoglycolipid(s) in the composition, wherein:

the sum contents of Pro-Hyp and Hyp-Gly relative to the total amount of collagen peptides contained in the collagen peptide containing composition are 0.01 to 10 wt %, and

the sphingoglycolipid(s) are incorporated in 0.01 to 100 parts by weight per 100 parts by weight of the collagen peptides.

(8) The method according to (7) wherein the collagen peptides have a weight average molecular weight of less than 5000.
(9) The method according to (7) or (8) wherein the sphingoglycolipid(s) are derived from a ceramide raw material and a ceramide raw material is incorporated in 0.01 to 200 parts by weight per 100 parts by weight of the collagen peptides.
(10) The method according to any one of (7) to (9) wherein the ceramide raw material is milk-derived ceramide.
(11) A composition containing collagen peptides and glycerophospholipids, wherein:

the collagen peptides are contained in 0.01 to 99.9 wt % of the total amount of the composition and the sum contents of Pro-Hyp and Hyp-Gly relative to the total amount of the collagen peptides are 0.01 to 10 wt %; and

in the glycerophospholipids, the content of a neutral glycerophospholipid is at least 1.5 times the content of an acidic glycerophospholipids on a weight basis, with the neutral glycerophospholipid being contained in 0.01 to 100 parts by weight per 100 parts by weight of the collagen peptides.

(12) The composition according to (11) wherein the collagen peptides have a weight average molecular weight of less than 5000.
(13) The composition according to (11) or (12) wherein the neutral glycerophospholipid is selected from the group consisting of phosphatidyl choline, phosphatidyl ethanolamine, and a combination thereof.
(14) The composition according to any one of (11) to (13) wherein the neutral glycerophospholipid is derived from a ceramide raw material and a ceramide raw material is incorporated in 0.01 to 200 parts by weight per 100 parts by weight of the collagen peptides.
(15) The composition according to (14) wherein the ceramide raw material is milk-derived ceramide.
(16) A composition containing collagen peptides and sphingoglycolipid(s), wherein:

the collagen peptides are contained in 0.01 to 99.9 wt % of the total amount of the composition and the sum contents of Pro-Hyp and Hyp-Gly relative to the total amount of the collagen peptides are 0.01 to 10 wt %; and

the sphingoglycolipid(s) are contained in 0.01 to 100 parts by weight per 100 parts by weight of the collagen peptides.

(17) The composition according to (16) wherein the collagen peptides have a weight average molecular weight of less than 5000.
(18) The composition according to (16) or (17) wherein the sphingoglycolipid(s) are derived from a ceramide raw material and a ceramide raw material is incorporated in 0.01 to 200 parts by weight per 100 parts by weight of the collagen peptides.
(19) The composition according to (18) wherein the ceramide raw material is milk-derived ceramide.

### Advantageous Effects of Invention

According to the present invention, the distinctive bitterness of collagen peptides can be reduced, advantageously making it possible for the collagen peptides to be ingested in a daily and continuous manner. As a result, the various effects possessed by the collagen peptides can be effectively displayed.

In addition, the skin improving effect of glycerophospholipids or sphingoglycolipids or materials containing these substances (e.g. a ceramide raw material) is exhibited, which is also advantageous in that the useful effects on the skin are enhanced.

### DESCRIPTION OF EMBODIMENTS

Hereinafter, embodiments of the present invention are described in detail.

Methods for Masking Bitterness

One embodiment of the present invention is a method for masking the bitterness derived from collagen peptides by incorporating glycerophospholipids. More specifically, the bitterness of collagen peptides is masked by incorporating glycerophospholipids comprising an acidic glycerophospholipid and a neutral glycerophospholipid in specified proportions.

Another embodiment of the present invention is a method for masking the bitterness derived from collagen peptides by incorporating sphingoglycolipid(s). More specifically, the bitterness of collagen peptides is masked by incorporating sphingoglycolipid(s) in a specified proportion relative to the collagen peptides.

<Collagen Peptides>

The term “collagen peptides” as used in the present invention generally refers to collagen proteins that are reduced in molecular weight. Collagen peptides can be used to prevent and/or ameliorate symptoms of the skin and they are capable of preventing or ameliorating symptoms of the skin such as, for example, lowered skin moisture retention and elasticity, reduced skin firmness and gloss, rough skin, wrinkles, and dullness.

Collagen peptides may be obtained by subjecting collagen or modified collagen such as gelatin to hydrolysis treatments as by enzyme, acid, alkali, etc.; they may also be artificial products of synthesis; one, two or more of these materials may be employed. Examples of collagen and gelatin include ones derived from animals such as cattle, pig and chicken or derived from fish; in particular, collagen proteins that are extracted from the connective tissues in the skin, bone, tendon, etc. of animals, as well as from fish skin and scale may be employed. Enzymes to be used to prepare collagen peptides may be of any types that are capable of cutting peptide bonds in collagen or gelatin and examples include collagenase, papain, bromelain, actinidin, ficin, cathepsin, pepsin, chymosin, trypsin, and enzyme preparations consisting of these enzymes in admixture. Examples of acids that may be used include hydrochloric acid, sulfuric acid, nitric acid, etc. Examples of alkalis that may be used include sodium hydroxide, calcium hydroxide, etc.

In the present invention, collagen peptides obtained by hydrolysis may be used as they are in the form of an aqueous solution or they may be dried or otherwise reduced to a powder form. Alternatively, the aqueous solution may be subjected to a commonly employed treatment for purification and then used either as such or in other forms including powder. Whichever of these forms is employed, the effects of the present invention will in no way be compromised.

The weight average molecular weights of collagen peptides to be used in the present invention may, for example, be less than 5,000, preferably less than 3,000, more preferably in the range of 100 to 3,000, even more preferably in the range of 300 to 2,000, and still more preferably in the range of 800 to 1,200. The weight average molecular weight of collagen peptides can be measured by known quantification methods such as HPLC and gel filtration. It should be noted here that the in vivo absorbability of collagen peptides generally decreases with the increasing weight average molecular weight and vice versa but collagen peptides with smaller weight average molecular weights present unwanted eating qualities that are characteristic of peptides. The present inventors have discovered that collagen peptides having the above-specified weight average molecular weights display particularly significant bitterness.

And the collagen peptides used in the present invention have high concentrations of Pro-Hyp (PO) and/or Hyp-Gly (OG); for instance, when measured in an aqueous solution containing 0.05 wt % collagen peptides, the content of PO is typically 100 nM or greater, preferably 200 nM or greater, more preferably 300 nM or greater whereas the content of OG is typically 200 nM or greater, preferably 300 nM or greater, more preferably 400 nM or greater, with the sum contents of PO and OG being typically 230 nM or greater, preferably 300 nM or greater, more preferably 500 nM or greater, even more preferably 700 nM or greater. The concentrations of PO and/or OG can be measured by known methods using such devices as LC/MS/MS. In the study that led to the accomplishment of the present invention, the present inventors have discovered that collagen peptides containing PO and/or OG at the above-specified concentrations present high degrees of bitterness.

And the contents of PO and OG in collagen peptides are such that relative to the total amount of collagen peptides, the PO content is typically 0.1 wt % or greater, preferably 0.15 wt % or greater, more preferably 0.20 wt % or greater whereas the OG content is typically 0.20 wt % or greater, preferably 0.30 wt % or greater, more preferably 0.50 wt % or greater, with the sum contents of PO and OG being typically 0.01 to 100 wt % or greater, preferably 0.05 to 90 wt % or greater, and more preferably 0.1 to 80 wt % or greater.

Collagen peptides may be used either as an extract or in purified form and it is preferred to use products with a purity of 50 wt % or greater, more preferably with a purity of 70 wt % or greater, and even more preferably with a purity of 90 wt % or greater. Commercial products of collagen peptide may also be used, as exemplified by COLLAGEN PEPTIDE HDL-50DR, COLLAPEP JB, COLLAGEN PEPTIDE SCP-5100, COLLAGEN PEPTIDE 800F, COLLAGEN PEPTIDE HDL-30DR, COLLAGEN PEPTIDE LCP and COLLAPEP PU (all manufactured by Nitta Gelatin Inc.)

The content of collagen peptides in the collagen peptide containing composition is difficult to specify uniquely since it varies with the type of the starting materials to be combined with them, their contents and other factors but it may range from 1 wt % to 99.9 wt %, preferably from 30 wt % to 99.9 wt %, and more preferably from 50 wt % to 90 wt %. And the dosage of the collagen peptide containing composition may be determined as appropriate for the age, body weight, health status and other conditions of the subject to which it is administered; consider, for example, the daily intake by a human adult and it is typically in the range from 10 mg to 100,000 mg, preferably from 500 mg to 15,000 mg, more preferably from 1,000 mg to 10,000 mg, which may be ingested or administered in a single dose or divided doses. The daily intake of PO and OG by a human adult is typically in the range from 0.1 mg to 200 mg, preferably from 0.5 mg to 100 mg, more preferably from 1 mg to 50 mg.

<Active Ingredients>

(Glycerophospholipids)

Complex lipids as a class of lipids are generally known as lipids that contain phosphates or sugars in their molecule, and they typically include phospholipids and glycolipids. And phospholipids include two types, glycerophospholipids and sphingophospholipids whereas glycolipids typically include sphingoglycolipids and glyceroglycolipids. It should be noted that the lipids as used in the present invention shall include not only the individual lipids as such but also their lyso forms.

The term “glycerophospholipids” as used in the present invention includes neutral glycerophospholipids and acidic glycerophospholipids; examples of neutral glycerophospholipids include phosphadityl choline (PC) (also known as lecithin) and phosphatidyl ethanolamine (PE), and examples of acidic glycerophospholipids include phosphadityl serine (PS), phosphadityl inositol (PI), and phosphadityl glycerol (PG).

The above-mentioned glycerophospholipids are not particularly limited in origin and they may be derived from natural products or they may be chemically synthesized; either an extract or a purified form may be employed; alternatively, a raw material containing more than one type of lipids (e.g. ceramide raw material) may also be used.

For instance, plant or animal derived raw material lecithins (e.g. milk/soy lecithin and egg yolk lecithin) may be used as glycerophospholipids. Lipid-containing raw materials available on the market include, for example, glycerophospholipid containing, milk-derived MILK CERAMIDE MC-5 (MEGMILK SNOW BRAND Co., Ltd.), a 40% phosphatidyl choline containing lecithin extract LIPOID S40 (Kenko Corporation) which is a phosphatidyl choline containing soy lecithin extract, and SERINE AID 50P (Kenko Corporation) which is a phosphatidyl serine containing soy lecithin extract.

(Containing Ratio of Neutral and Acidic Glycerophospholipids)

The present inventors have found that by adding neutral glycerophospholipids in proportions greater than a certain level of acidic glycerophospholipids, the bitterness of the above-specified collagen peptides can be effectively masked. To state specifically, by adding neutral glycerophospholipids in amounts that are at least 1.5 times, preferably at least 1.6 times, more preferably at least 1.7 times, the amount of acidic glycerophospholipids on a weight basis, it becomes possible to mask the bitterness of collagen peptides.

(How Much to Incorporate Neutral Glycerophospholipids)

The present inventors have further found that by incorporating neutral glycerophospholipids in specified proportions relative to the weight of collagen peptides, bitterness masking is also possible. The amount of neutral glycerophospholipids to be incorporated is not particularly limited but the bitterness of the collagen peptide containing composition can be masked by adding neutral glycerophospholipids typically in 0.01 to 100 parts by weight, preferably in 0.02 to 10 parts by weight, more preferably in 0.03 to 1 part by weight, per 100 parts by weight of collagen peptides.

(Sphingoglycolipids)

The term “sphingoglylcolipids” as used in the present invention refers to complex lipids based on sphingosine to which a fatty acid binds, with a sugar being further linked by a glycoside bond, and examples of sphingoglylcolipids include glucosyl ceramide and galactosyl ceramide.

Sphingoglylcolipids may be of animal or plant origin but sphingoglylcolipids derived from plants (e.g. wheat, soybean, konjac potato) are preferably used. And sphingoglylcolipids may be used either as a purified product or as an extract, typically with a purity of 0.01 wt % or greater, preferably with a purity of 0.1 wt % or greater, and more preferably with a purity of 1.0 wt % or greater. If desired, commercial products may also be used and examples include PHYTOCERAMIDE (10%) (ICHIMARU PHARCOS Co., Ltd.) which is a ceramide containing rice extract.

Sphingoglylcolipids are typically incorporated in 0.01 to 100 parts by weight, preferably in 0.02 to 10 parts by weight, more preferably in 0.03 to 1 part by weight, per 100 parts by weight of collagen peptides, whereby it becomes possible to mask the bitterness of collagen peptides.

(Ceramide Raw Material)

“Ceramide” in the narrow sense is generally a type of sphingolipids consisting of a fatty acid in amide linkage to sphingosine. Ceramide in the narrow sense is abundant in the keratin and is known to be deeply involved in the expression of skin's protection and barrier functions. Substances having other structures are sometimes called ceramide and “ceramides” widely used in the esthetic industry include lipids such as sphingomyelins in addition to the above-described ceramide in the narrow sense.

The “ceramide raw material” as used in the present invention refers to raw materials that contain the various glycerophospholipids or sphingoglycolipids listed above or combinations thereof. The ceramide raw material may contain not only natural ceramides but also pseudo-ceramides that are similar to natural ceramides in structure and properties, as well as extracts or derivatives from these components. The ceramide raw material is not particularly limited in origin and animal ceramide raw materials (e.g. milk ceramide) or plant ceramide raw materials such as from rice, wheat, soybean and potato may be employed. The milk-derived ceramide here mentioned is rich in glycerophospholipids whereas plant-derived ceramides contain sphingoglycolipids as a major component. The ceramide raw material may be used either as a purified product or as an extract, preferably with a purity of 0.01 wt % or greater, more preferably with a purity of 0.1 wt % or greater, and even more preferably with a purity of 1.0 wt % or greater. Examples of commercially available ceramide raw materials that may be used in the present invention include MILK CERAMIDE MC-5 (MEGMILK SNOW BRAND Co., Ltd.) which is a ceramide containing whey powder and PHYTOCERAMIDE (10%) (ICHIMARU PHARCOS Co., Ltd.) which is a ceramide containing rice extract.

The present inventors have found that by containing the ceramide raw material in a specified weight with respect to collagen peptides, the bitterness can be masked. To give specific but non-limiting values, the ceramide raw material may be contained in 0.01 to 200 parts by weight, preferably in 0.02 to 100 parts by weight, and more preferably in 0.03 to 10 parts by weight, per 100 parts by weight of collagen peptides, whereby it becomes possible to mask the bitterness of the collagen peptide containing composition.

<Other Ingredients>

Aside from the active ingredients described above, known additives may be incorporated in the collagen peptide containing composition. Although the applicable additives are not particularly limited, those which are commonly used for oral ingestion are preferred and examples that can be used include excipients, binders, disintegrants, lubricants, antiseptics, flavoring agents, aroma corrigents, coloring agents, flavors, etc. and any raw materials can be used that are known to have skin improving effects.

The collagen peptide containing composition may have other beautifying or health promoting components incorporated in combination with the active ingredients. The components that can be used in combination with the active ingredients are not particularly limited and examples include elastin, proteoglycan, hyaluronic acid, lactobacilli, vitamins (e.g. vitamin C), minerals (e.g. calcium), plant extracts, etc. Components that are known to possess a skin improving effect like collagen may further be incorporated, and ingredients that are known to have the fibroblasts proliferation promoting effect are exemplified by: dried products or extracts of plants and algae such as chlorella, aloe barbadensis, rice, jujube, Alpinia zerumbet, Curcuma amada, Ampelopsis brevipedunculata, Adiantum capillus-veneris, Nelumbo nucifera germ, sesame, pepper, Angelica acutiloba, Houttuynia cordata, Lonicera caerulea var. emphyllocalyx fruit, Mallotus philippensis (Lam.) Muell. Arg., algae (Caulerpa racemosa), Rubus ellipticus, and Coix lacryma-jobi var. ma-yuen; catechins, imino-group containing peptides, α-lipoic acid and its derivatives such as salts, esters, amides, etc., dihydrolipoic acid and its derivatives, chitin hydrolyzates, N-acetyl-D-glucosamine and its oligomers.

<Form and Dosage>

The form of the collagen peptide containing composition is not particularly limited but it is preferably in an oral dosage form, typically in the form of oral preparations such as granules, tablets, capsules, and liquids/solutions.

<Production Method>

While the collagen peptide containing composition can be produced in the usual manner, the glycerophospholipids and sphingoglycolipids may be incorporated either as such or after being processed into a solid, paste or liquid form. If desired, other components may optionally be incorporated. The contents and proportions of those and other components are as described above.

For example, collagen peptides, glycerophospholipids or sphingoglycolipids and raw material containing these components (e.g. ceramide raw material) may optionally be mixed with excipients (e.g. glucose, dextrin, lactose, starch or its processed products, and cellulose powder), vitamins, minerals, fats and oils from animals, plants, fishes and shells, proteins (e.g. animal-, plant- or yeast-derived proteins, and their hydrolyzates), saccharides, pigments, flavors, antioxidants, surfactants, other additives, a variety of ingredients with nutrient function claims, as well as casein, etc. in powder or extract form, and the mixtures are either processed into powder, granular, pellet, tablet and other forms; if desired, these mixtures in liquid form may be coated with gelatin, sodium alginate, carboxymethyl cellulose or other coating agents to mold capsules.

Compositions

A further mode of the present invention is a composition containing collagen peptides and glycerophospholipids. In the composition of this mode, neutral glycerophospholipids are contained in a specific proportion with respect to collagen peptides and the proportion between neutral and acidic glycerophospholipids is set at a specific value, whereby the bitterness of collagen peptides can be masked.

Another mode of the present invention is a composition containing collagen peptides and sphingoglycolipids. In the composition of this mode, sphingoglycolipids are contained in a specific proportion with respect to collagen peptides, whereby the bitterness of collagen peptides can be masked.

The content of collagen peptides in the composition is difficult to specify uniquely since it varies with the type of the starting materials to be combined with them, their contents and other factors but it may range from 1 wt % to 99.9 wt %, preferably from 30 wt % to 99.9 wt %, and more preferably from 50 wt % to 90 wt %. And the dosage of the collagen peptide containing composition may be determined as appropriate for the age, body weight, health status and other conditions of the subject to which it is administered; consider, for example, the daily intake by a human adult and it is typically in the range from 10 mg to 100,000 mg, preferably from 500 mg to 15,000 mg, more preferably from 1,000 mg to 10,000 mg, which may be ingested or administered in a single dose or divided doses. The daily intake of PO or OG by a human adult is typically in the range from 0.1 mg to 200 mg, preferably from 0.5 mg to 100 mg, more preferably from 1 mg to 50 mg.

The ratio at which the neutral glycerophospholipid is to be incorporated in the composition is not particularly limited but it is typically contained in 0.01 to 100 parts by weight, preferably in 0.02 to 10 parts by weight, more preferably in 0.03 to 1 part by weight per 100 parts by weight of collagen peptides. The containing ratios of the neutral and acidic glycerophospholipids in the glycerophospholipids to be used in the composition are such that the neutral glycerophospholipid is at least 1.5 times, preferably at least 1.6 times, more preferably at least 1.7 times, the acidic glycerophospholipid on a weight basis.

The amount of sphingoglycolipids to be incorporated in the composition is such that sphingoglycolipids are in 0.01 to 100 parts by weight, preferably in 0.02 to 10 parts by weight, more preferably in 0.03 to 1 part by weight per 100 parts by weight of collagen peptides.

The glycerophospholipids or sphingoglycolipids to be incorporated in the composition may derive from the ceramide raw material. In this case, the composition may contain the ceramide raw material in a specified weight relative to collagen peptides. For example, the ceramide raw material is contained in 0.01 to 200 parts by weight, preferably in 0.02 to 100 parts by weight, more preferably in 0.03 to 10 parts by weight, per 100 parts by weight of collagen peptides.

Regarding the type of components to be used in the compositions of the modes under consideration, their recipes, forms and dosages, as well as the methods for their production, the contents of the disclosure made about the bitterness masking method may be applied as such.

### EXAMPLES

On the following pages, the present invention will be described in greater detail based on Examples, to which the present invention is by no means limited.

Raw Materials Used

The raw materials used in the following Examples are listed in Tables 1 and 2. Here, unless otherwise indicated (to the effect that the weights of raw materials of their own are shown), the amounts indicated for collagen peptides, glycerophospholipids and sphingoglycolipids used in this EXAMPLES section were obtained by calculation based the contents of the components contained in the raw materials shown in Table 1.

TABLE 1
<Raw materials Used: Ceramide Raw Material/Glycerophospholipids/Sphingoglycolipids>

| Raw | Product name | Description | |
|---|---|---|---|
| Materials | (Manufacturer) | (Origin) | Components and their contents |
| 1 | MILK CERAMIDE MC-5 | Ceramide-containing | Phosphatidyl choline (PC): 6.6% |
| | (MEGMILK SNOW | whey powder | Phosphatidyl ethanolamine (PE): |
| | BRAND Co., Ltd.) | (milk-derived) | 10.5% |
| | | | Phosphatidyl serine (PS): 4.5% |
| | | | Phosphatidyl inositol (PI): 5.3% |
| 2 | 40% Phosphatidyl choline | Soy lecithin extract | Phosphatidyl choline: 40% contained |
| | containing lecithin extract, | (soybean-derived) | (found: 43.8%) |
| | LIPOID S40 | | |
| | (Kenko Corporation) | | |
| 3 | SERINE AID 50P | Soy lecithin extract | Phosphatidyl choline: 50% contained |
| | (Kenko Corporation) | (soybean-derived) | (found: 51.0%) |
| 4 | PHYTOCERAMIDE (10%) | Ceramide containing | Sphingoglycolipid: more than 10.0% |
| | (ICHIMARU PHARCOS | rice extract | contained (found: 10.8%) |
| | Co., Ltd) | (rice-derived) | |

TABLE 2
<Raw Materials Used: Collagen Peptides>

| | | Weight | PO-OG sum | | |
|---|---|---|---|---|---|
| | | average | contents | Components | |
| Raw | Product name | molecular | (nM in 0.05 wt | and their | |
| Materials | (Manufacturer) | weight | % aq. sol.) | contents | Origin |
| 5 | Collagen peptide | 5000 | 155 | Collagen | Derived from |
| | HDL-50DR | | | peptide 100% | acid treated |
| | (Nitta Gelatin Inc.) | | | | fish scale and |
| | | | | | skin |
| 6 | Collagen peptide | 5000 | 230 | Collagen | Derived from |
| | SCP-5100 | | | peptide 100% | acid treated |
| | (Nitta Gelatin Inc.) | | | | pig skin |
| 7 | Collagen peptide | 3000 | 209 | Collagen | Derived from |
| | HDL-30DR | | | peptide 100% | acid treated |
| | (Nitta Gelatin Inc.) | | | | fish scale and |
| | | | | | skin |
| 8 | Collapep PU | 1200 | 15768 | Collagen | Derived from |
| | (Nitta Gelatin Inc.) | | | peptide 100% | acid treated |
| | | | | | pig skin |
| 9 | Collagen peptide | 1200 | 16835 | Collagen | Derived from |
| | Sun-12 | | | peptide 100% | acid treated |
| | (Nitta Gelatin Inc.) | | | | fish scale and |
| | | | | | skin |
| 10 | Collagen peptide | 800 | 12120 | Collagen | Derived from |
| | Sun-8 | | | peptide 100% | acid treated |
| | (Nitta Gelatin Inc.) | | | | fish scale and |
| | | | | | skin |

### Example 1

### Masking Effect of Ceramide Raw Material Against the Bitterness of Collagen Peptides

### <Evaluating the Bitterness of Collagen Peptides>

To confirm the bitterness masking effect of raw materials, the degree of bitterness from collagen peptides was first evaluated.

(Method of Evaluation)

Sensory evaluation was made using collagen peptides of varying weight average molecular weights and different origins (Raw Materials 5-10 in Table 2); specifically, the respective collagen peptides in powder form (0.5 g) were put into the mouth for evaluating the degree of bitterness from the collagen peptides alone. In addition, the PO (Pro-Hyp) and OG (Hyp-gly) contents in each collagen peptide were measured and the correlation between their contents and bitterness was also evaluated. The sensory evaluation was performed by five trained expert panelists and the results were rated on a 10-score scale ranging from 0 (no bitterness sensed) to 10 (utmost bitterness sensed). The degree of bitterness from Raw Materials 5-11 was evaluated and the mean values for the 5 expert panelists are shown in Table 3.

(Results of Evaluation)

As Table 3 shows, collagen peptides having weight average molecular weights of 5000 and less developed bitterness which increased with decreasing weight average molecular weight, and the bitterness became markedly intense when the weight average molecular weight was less than 3000. From the results with Samples 4 and 5 having collagen peptides of the same weight average molecular weight, a collagen peptide having increased PO and OG contents (Raw Material 9) was shown to have stronger bitterness. As it turned out, bitterness was particularly strong when the sum contents of PO and OG as measured in a 0.05 wt % collagen peptide aqueous solution were 230 nM and greater.

TABLE 3
<Evaluation of Bitterness from Samples Containing Collagen Peptides and Ceramide Raw Materials>

| | Collagen peptide | | | | | | |
|---|---|---|---|---|---|---|---|
| | Weight Average | PO | OG | PO + OG | Degree of | | |
| | Molecular weight | (nM in | (nM in | (nM in | bitterness | Degree of | |
| | (MW) | 0.05% aq. | 0.05% aq. | 0.05% | from collagen | sample's | Degree of |
| Sample*¹ | (Raw Materials) | sol.) | sol.) | aq. sol.)) | peptide alone | bitterness | masking*² |
| 1 | 5000 | 79 | 76 | 155 | 3.4 | 3.2 | 0.2 |
| | (Raw Material 5) | | | | | | |
| 2 | 5000 | 41 | 189 | 230 | 2.4 | 1.4 | 1.0 |
| | (Raw Material 6) | | | | | | |
| 3 | 3000 | 76 | 133 | 209 | 5.8 | 4.6 | 1.2 |
| | (Raw Material 7) | | | | | | |
| 4 | 1200 | 315 | 15453 | 15768 | 7.8 | 5.4 | 2.4 |
| | (Raw Material 8) | | | | | | |
| 5 | 1200 | 352 | 16483 | 16835 | 8.6 | 4.6 | 4.0 |
| | (Raw Material 9) | | | | | | |
| 6 | 800 | 9292 | 2828 | 12120 | 10.0 | 6.0 | 4.0 |
| | (Raw Material 10) | | | | | | |

*¹Collagen peptide and ceramide raw material (Raw Material 1) were mixed at a weight ratio of 1:1.
*²Calculation was made by the formula [Degree of masking] = [Degree of bitterness from collagen peptide alone] − [degree of bitterness from sample].

<Evaluating the Masking Effect of Ceramide Raw Material>

The milk ceramide (Raw Material 1 in Table 1) was evaluated for its ability to mask the above-measured bitterness of collagen peptides. As Table 1 shows, the milk ceramide as Raw Material 1 under test contained neutral glycerophospholipids in an amount more than 1.5 times the amount of acidic glycerophospholipids. The glycerophospholipids contained in the milk ceramide as Raw Material 1 had the following recipe:

Neutral glycerophospholipids: 17.1%

phosphatidyl choline (PC): 6.6%

phosphatidyl ethanolamine (PE): 10.5%

Acidic glycerophospholipids: 9.8%

phosphadityl serine (PS): 4.5%

phosphatidyl inositol (PI): 5.3%

[Weight of neutral glycerophospholipids]/[Weight of acidic glycerophospholipids]=1.74

(Method of Evaluation)

The collagen peptides as Raw Materials 5-10 were each weighed and mixed with Raw Material 1 (milk ceramide) at a ratio of 1:1 to prepare Samples 1-6 in powder form. Samples 1-6 each weighing 0.5 g were subjected to sensory evaluation by five expert panelists in accordance with the same method as used above to evaluate the bitterness of collagen peptides alone. The mean values of bitterness from Samples 1-6 are shown in Table 3. The degree of masking was determined by subtracting the mean values of bitterness of Samples 1-6 from the mean values of bitterness of collagen peptides alone and the results are shown in Table 3.

(Results of Evaluation)

As Table 3 shows, the ceramide raw material turned out to provide high masking effects when the weight average molecular weight of collagen peptides was 5000 or less. The masking effect was marked against collagen peptides having weight average molecular weights of less than 3000. It was also revealed that the masking effect of the ceramide raw material was even higher in the case of using intensely bitter collagen peptides in which the sum contents of PO and OG as measured in a 0.05 wt % collagen peptide aqueous solution were 230 nM and greater.

### Example 2

### Evaluation of the Ratio Between Collagen Peptides and Neutral or Acidic Glycerophospholipid to be Incorporated

Using high concentrations of a neutral or acidic glycerophospholipid, an evaluation was made to investigate the ratios between collagen peptides and the neutral or acidic glycerophospholipid to be incorporated in order to effectively mask the bitterness of collagen peptides.

<Method of Evaluation>

In accordance with the method of Example 1, a collagen peptide (Raw Material 9 in Table 2) was mixed with a neutral glycerophospholipid (Raw Material 2 in Table 1) or an acidic glycerophospholipid (Raw Material 3 in Table 1) at the ratios indicated in Table 4 to prepare Samples 7-13 which were subjected to sensory evaluation, and the results are shown in Table 4.

<Results of Evaluation>

First of all, as Table 4 shows, the higher the ratio of glycerophospholipids to collagen peptides, the higher the masking effect and the use of the neutral glycerophospholipid turned out to be significant. It was also found that the neutral glycerophospholipid exhibited its effect at a concentration that was about one thousandth of the concentration of the acidic glycerophospholipid.

As one can see from the data of Table 4, the masking effect was significant when the ratio between collagen peptides and the glycerophospholipid raw material was 1/0.00025 or above and the ratio between collagen peptides and the glycerophospholipid alone was 1/0.0001 or above, clearly indicating that the neutral glycerophospholipid has a greater masking effect than the acidic glycerophospholipid.

TABLE 4
<Evaluating the ratio between collagen peptides and glycerophospholipids incorporated>

| | | Mixing ratio between collagen peptides | | |
|---|---|---|---|---|
| | | and glycerophospholipid raw material | | |
| | | based on weight of | based on weight of | |
| | Glycerophospholipid Raw | glycerophospholipid | glycerophospholipid | Degree of |
| Sample | Material | Raw Material | (net weight) | masking*³ |
| 7 | Raw Material 2 | 1/0.000025 | 1/0.00001 | 0.0 |
| 8 | (phosphatidyl choline (neutral | 1/0.00025 | 1/0.0001 | 2.4 |
| 9 | glycerophospholipid) | 1/0.0025 | 1/0.001 | 4.6 |
| 10 | containing raw material) | 1/0.025 | 1/0.01 | 5.2 |
| 11 | | 1/0.25 | 1/0.1 | 7.0 |
| 12 | | 1/2.5 | 1/1 | 8.0 |
| 13 | Raw Material 3 | 1/0.000025 | 1/0.0000125 | 0.0 |
| 14 | (phosphatidyl serine (acidic | 1/0.00025 | 1/0.000125 | 0.0 |
| 15 | glycerophospholipid) | 1/0.0025 | 1/0.00125 | 1.2 |
| 16 | containing raw material) | 1/0.025 | 1/0.0125 | 1.6 |
| 17 | | 1/0.25 | 1/0.125 | 2.4 |
| 18 | | 1/2.5 | 1/1.25 | 5.4 |

*³Calculation was made by the formula [Degree of masking] = [Degree of bitterness from collagen peptide alone] − [degree of bitterness from sample].

### Example 3

### Investigating Optimum Proportions of Neutral and Acidic Glycerophospholipids

The ratio of glycerophospholipids incorporated in raw materials was varied to investigate optimum proportions of neutral and acidic glycerophospholipids that showed an effect for masking the bitterness of collagen peptides.

<Method of Evaluation>

Ceramide raw materials were prepared by blending two raw materials having high glycerophospholipid contents (Raw Materials 2 and 3 in Table 1) in the proportions indicated in Table 5. The resulting ceramide raw materials were mixed with collagen peptides (Raw Material 9 in table 2) to prepare Samples 19-23; each sample was evaluated for its masking effect and the results are shown in Table 5. Sample preparation and sensory evaluation were made in accordance with the methods of Example 1.

<Results>

As Table 5 shows, the masking effect was clearly exhibited when the proportion of the neutral glycerophospholipid was higher than that of the acidic glycerophospholipid. The inventors also confirmed that the masking effect was significant when the neutral glycerophospholipid was two or more times as heavy as the acidic glycerophospholipid. From this data, it was confirmed that the effect for masking the bitterness of collagen peptides became more marked by further increasing the proportion of the neutral glycerophospholipid over Raw Material 1, a ceramide raw material used in Example 1.

TABLE 5
<Evaluation of the proportions of neutral and acidic glycerophospholipids>

| | Recipe of ceramide raw material | | | | | |
|---|---|---|---|---|---|---|
| | Raw Material 2 | | Raw Material 3 | | | |
| | (phosphatidyl | | (phosphatidyl | | | |
| | choline (neutral | | serine (acidic | | | |
| | glycerophospholipid) | | glycerophospholipid) | | | |
| | containing raw | phosphatidyl | containing raw | phosphatidyl | [PC weight]/ | Degree of |
| Sample*⁴ | material) | choline (PC) | material) | serine (PS) | [PS weight] | masking*⁵ |
| 19 | 1.000000 | 0.400000 | 1.000000 | 0.500000 | 0.8 | 0.4 |
| 20 | 1.000000 | 0.400000 | 0.400000 | 0.200000 | 2.0 | 2.0 |
| 21 | 1.000000 | 0.400000 | 0.325000 | 0.162500 | 2.5 | 3.2 |
| 22 | 1.000000 | 0.400000 | 0.200000 | 0.100000 | 4.0 | 4.8 |
| 23 | 1.000000 | 0.400000 | 0.100000 | 0.050000 | 8.0 | 7.4 |

*⁴Collagen peptides (Raw Material 9) and ceramide raw material were mixed at a weight ratio of 1:1.
*⁵Calculation was made by the formula [Degree of masking] = [Degree of bitterness from collagen peptide alone] − [degree of bitterness from sample].

### Example 4

### Masking Effect of Sphingoglycolipids

Sphingoglycolipids, a typical ceramide raw material (and derived from plant ceramides) were used to evaluate their effect for masking the bitterness of collagen peptides.

<Method of Evaluation>

Collagen peptides (Raw Material 9 in Table 2) and sphingoglycolipids (Raw Material 4 in Table 1) were mixed at the proportions indicated in Table 6 to prepare Samples 24-29, which were subjected to sensory evaluation. Sample preparation and sensory evaluation were made in accordance with the methods of Example 1.

<Results of Evaluation>

As Table 6 shows, the sphingoglycolipids turned out to be at least comparable to the neutral glycerophospholipid in their effect for masking the bitterness of collagen peptides. Specifically, a significant masking effect was achieved when the ratio of collagen peptides to ceramide raw material (as composition) was 1/0.001 or greater, and the ratio of collagen peptides to sphingoglycolipids alone was 1/0.0001 or greater. It was also revealed that the masking effect became more significant by incorporating the sphingoglycolipids in increased amounts. Thus, it was clear that in addition to the milk ceramide as an animal-derived ceramic raw material, the sphingoglycolipids as a major component of plant-derived ceramide raw materials exhibited a high masking effect against the bitterness of collagen peptides.

TABLE 6
<Evaluation of the masking effect of sphingoglycolipids>

| | Mixing ratio of collagen peptides (Raw | | |
|---|---|---|---|
| | Material 9) and ceramide raw material | | |
| | [collagen peptide weight]/[ceramide weight] | | |
| | based on weight of | | |
| | Raw Material 4 (as | based on weight of | Degree of |
| Sample | composition) | sphingoglycolipids (as such) | masking*⁶ |
| 24 | 1/0.0001 | 1/0.00001 | 0.0 |
| 25 | 1/0.001 | 1/0.0001 | 3.8 |
| 26 | 1/0.01 | 1/0.001 | 4.8 |
| 27 | 1/0.1 | 1/0.01 | 5.8 |
| 28 | 1/1 | 1/0.1 | 7.0 |
| 29 | 1/10 | 1/1 | 9.0 |

*⁶Calculation was made by the formula [Degree of masking] = [Degree of bitterness from collagen peptide alone] − [degree of bitterness from sample].

Production 1: Powder Ready for Dissolving Just Before Use

Collagen, ceramide raw material, elastin, proteoglycan, vitamin C, dextrin and other excipients were weighed in the respective amounts indicated in Table 7 below and mixed uniformly to prepare samples in powder form (6.5 g) which were ready for dissolving just before use. The resulting powder was dissolved in water and every sample, showing good dispensability, was satisfactory as a beverage ready for dissolving just before drinking.

TABLE 7
<Recipe for powder ready for dissolving just before use>

| Ingredients | Amount (mg) | Proportions (wt %) |
|---|---|---|
| Collagen peptides | 5000.0 | 76.92 |
| Ceramide Raw Material (neutral | 24.0 | 0.37 |
| glycerophospholipids | | |
| 17.1% incorporated) | | |
| Elastin peptide | 1.0 | 0.02 |
| Proteoglycan | 0.5 | 0.01 |
| Vitamin C | 70.0 | 1.08 |
| Dextrin and other excipients | 1404.5 | 21.60 |
| Total | 6500.0 | 100.00% |

Production 2: Tablet

A mixture of the recipe identified below was granulated and molded in the usual manner to produce tablets.

TABLE 8
<Recipe for tablet>

| Ingredients | Amount (mg) | Proportions (wt %) |
|---|---|---|
| Collagen peptides | 5000.0 | 76.92 |
| Ceramide Raw Material (neutral | 24.0 | 0.37 |
| glycerophospholipids | | |
| 17.1% incorporated) | | |
| Elastin peptide | 1.0 | 0.02 |
| Proteoglycan | 0.5 | 0.01 |
| Vitamin C | 70.0 | 1.08 |
| Cellulose and other excipients | 1404.5 | 21.60 |
| Total | 6500.00 | 100.00% |

### INDUSTRIAL APPLICABILITY

Glycerophospholipids comprising neutral glycerophospholipids and acidic glycerophospholipids in specified proportions are incorporated or sphingoglycolipids are incorporated in a specified proportion relative to collagen peptides, whereby the bitterness of collagen peptides is masked, enabling them to be ingested freely and easily.

1: A method for masking the bitterness of a collagen peptide containing composition, which comprises incorporating glycerophospholipids in the composition, wherein:
the sum contents of Pro-Hyp and Hyp-Gly relative to the total amount of the collagen peptides contained in the collagen peptide containing composition are 0.01 to 10 wt %, and
in the glycerophospholipids, the content of a neutral glycerophospholipid is at least 1.5 times the content of an acidic glycerophospholipid on a weight basis.

2: The method according to claim 1 wherein the collagen peptides have a weight average molecular weight of less than 5000.

3: The method according to claim 1 wherein the neutral glycerophospholipid is incorporated in 0.01 to 100 parts by weight per 100 parts by weight of the collagen peptides.

4: The method according to claim 1 wherein the neutral glycerophospholipid is selected from the group consisting of phosphatidyl choline, phosphatidyl ethanolamine, and a combination thereof.

5: The method according to claim 1 wherein the neutral glycerophospholipid is derived from a ceramide raw material and a ceramide raw material is incorporated in 0.01 to 200 parts by weight per 100 parts by weight of the collagen peptides.

6: The method according to claim 5 wherein the ceramide raw material is milk-derived ceramide.

7: A method for masking the bitterness of a collagen peptide containing composition, which comprises incorporating sphingoglycolipid(s) in the composition, wherein:
the sum contents of Pro-Hyp and Hyp-Gly relative to the total amount of collagen peptides contained in the collagen peptide containing composition are 0.01 to 10 wt %, and
the sphingoglycolipid(s) are incorporated in 0.01 to 100 parts by weight per 100 parts by weight of the collagen peptides.

8: The method according to claim 7 wherein the collagen peptides have a weight average molecular weight of less than 5000.

9: The method according to claim 7 wherein the sphingoglycolipid(s) are derived from a ceramide raw material and a ceramide raw material is incorporated in 0.01 to 200 parts by weight per 100 parts by weight of the collagen peptides.

10: The method according to claim 7 wherein the ceramide raw material is milk-derived ceramide.

11: A composition containing collagen peptides and glycerophospholipids, wherein:
the collagen peptides are contained in 0.01 to 99.9 wt % of the total amount of the composition and the sum contents of Pro-Hyp and Hyp-Gly relative to the total amount of the collagen peptides are 0.01 to 10 wt %; and
in the glycerophospholipids, the content of a neutral glycerophospholipid is at least 1.5 times the content of an acidic glycerophospholipids on a weight basis, with the neutral glycerophospholipid being contained in 0.01 to 100 parts by weight per 100 parts by weight of the collagen peptides.

12: The composition according to claim 11 wherein the collagen peptides have a weight average molecular weight of less than 5000.

13: The composition according to claim 11 wherein the neutral glycerophospholipid is selected from the group consisting of phosphatidyl choline, phosphatidyl ethanolamine, and a combination thereof.

14: The composition according to claim 11 wherein the neutral glycerophospholipid is derived from a ceramide raw material and a ceramide fee is incorporated in 0.01 to 200 parts by weight per 100 parts by weight of the collagen peptides.

15: The composition according to claim 14 wherein the ceramide raw material is milk-derived ceramide.

16: A composition containing collagen peptides and sphingoglycolipid(s), wherein:
the collagen peptides are contained in 0.01 to 99.9 wt % of the total amount of the composition and the sum contents of Pro-Hyp and Hyp-Gly relative to the total amount of the collagen peptides are 0.01 to 10 wt %; and
the sphingoglycolipid(s) are contained in 0.01 to 100 parts by weight per 100 parts by weight of the collagen peptides.

17: The composition according to claim 16 wherein the collagen peptides have a weight average molecular weight of less than 5000.

18: The composition according to claim 16 wherein the sphingoglycolipid(s) are derived from a ceramide raw material and a ceramide raw material is incorporated in 0.01 to 200 parts by weight per 100 parts by weight of the collagen peptides.

19: The composition according to claim 18 wherein the ceramide raw material is milk-derived ceramide.
